(19) United States
(12) Patent Application Publication
(10) Pub. No.: US 2007/0093560 A1
(43) Pub. Date: Apr. 26, 2007

(54) Process for the holding operation of a Fischer-Tropsch synthesis

(75) Inventors: Anton Althapp, Oberschona (DE); Bodo M. Wolf, Mindelheim (DE); Dietmar Ruger, Bannewitz-Goppeln (DE); Olaf Schulze, Tuttendorf (DE)

Correspondence Address: FOLEY AND LARDNER LLP;SUITE 500, 3000 K STREET NW, WASHINGTON, DC, 20007, US

(73) Assignee: CHOREN INDUSTRIES GmbH

(21) Appl. No.: 11/584,755

(22) Filed: Oct. 23, 2006

(30) Foreign Application Priority Data
Oct. 21, 2005 (DE) 10 2005 050 526.0

(51) Int. Cl. C07C 27/26

(52) U.S. Cl. 518/726

(57) ABSTRACT

During required change-over to a holding operation in a Fischer-Tropsch synthesis, the reactor is not depressurized after cutting off the supply of fresh synthesis gas to the Fischer-Tropsch reactor, and the temperature is not reduced to below the temperature below which a Fischer-Tropsch reaction no longer takes place. Instead, the reactor is charged with an inert gas, e.g., kept in a vessel at a pressure higher than the operating pressure of the Fischer-Tropsch synthesis, until the reacting components have been flushed out from the Fischer-Tropsch system. While maintaining the pressure and the temperature, the reactor is thus merely inerted for a few seconds, sufficient to interrupt the Fischer-Tropsch reaction. Catalyst damage is avoided, and the liquid phase situation on the catalyst is at least essentially unchanged, facilitating subsequent re-commissioning of the catalyst.

### CROSS-REFERENCE TO RELATED PATENT APPLICATIONS

The right of foreign priority is claimed under 35 U.S.C. §119(a) based on Federal Republic of Germany Application No. 10 2005 050 526.0, filed Oct. 21, 2005, the entire contents of which, including the specification, drawings, claims and abstract, are incorporated herein by reference.

### BACKGROUND OF THE INVENTION

The invention relates to a process for carrying out an emergency start operation (due to an emergency stop) in a Fischer-Tropsch synthesis (FTS) utilizing a porous catalyst, and also to a facility for producing synthesis gas from combustion fuels.

One field of application of the invention resides in the production of synthetic hydrocarbons, such as gasoline, diesel fuel and wax, by means of a Fischer-Tropsch synthesis in association with a facility for the production of synthesis gas from combustion fuels.

The increasing reduction in the availability of liquid fuels, due to rising demand and the medium-term finite nature of petroleum, is leading to the increased use of the Fischer-Tropsch synthesis for manufacturing liquid hydrocarbons by the route of gas production, not only from combustion fuels such as natural gas and coal, which have a prospect of remaining available longer than petroleum, but also from renewable raw materials.

As early as in the 1930's in Germany and in the 1950's in South Africa, the Fischer-Tropsch synthesis (which had been developed in the 1920's) was operated in the form of coal gasification, due to lack of availability of liquid fuels. In the last two decades, several mineral oil companies have also increasingly worked on the development and the industrial application of a process combining gasification of natural gas and/or petroleum-by product gas and the synthesis of fuel. The state of art in this respect is defined by a Shell Oil Co. plant at Bintulu, Malaysia.

A complete facility for the Fischer-Tropsch synthesis comprises several units with at least the following process steps:
gas production for the production of an H₂-CO gas mixture with one or more other gas components, such as CO₂, CH₄, N₂, apart from catalyst poisons,
gas purification,
gas conditioning for producing the desired synthesis parameters, such as the H₂-CO ratio, among others,
if necessary, compression to produce the anticipated operating pressure in the synthesis, if the gas production and synthesis are not operated at the same pressures,
complete purification for the removal of residual catalyst poisons,
and the actual Fischer-Tropsch synthesis, to which further industrial systems such as a cracking plant and/or distillation plant may be connected downstream.

In the case of problems during the Fischer-Tropsch synthesis and the upstream process steps, as well as in the case of power failure, it may be necessary to shut down the synthesis in order not to damage the catalyst.

In the case of a failure of the synthesis gas supply to the Fischer-Tropsch reactor, the remaining amount of gas in the Fischer-Tropsch reactor leads to a condition in which the permissible reaction temperature may be exceeded, i.e., the reaction becomes a runaway reaction, is no longer kept under control and thus leads to the catalyst being damaged as well as inactivated.

In the case of problems which may lead to a loss of control of the strongly exothermal Fischer-Tropsch reaction, the Fischer-Tropsch synthesis can be rapidly changed over to a safe state. For this purpose, the Fischer-Tropsch synthesis is cut off from the supply of fresh synthesis gas, it is depressurized and its temperature is reduced as rapidly as possible to values at which no further Fischer-Tropsch reaction takes place, and it is usually scavenged with nitrogen. Even in this procedure, depending on the velocity of depressurizing and cooling, damage to the catalyst may occur which, depending on the level of damage, may go as far as leading to the replacement of the catalyst. If the process is shut down appropriately slowly, damage can be minimized. The damage to the catalyst may be of a mechanical nature and may also be manifested in a reduction of the catalytic effectiveness.

After eliminating the problem, a lengthy recommissioning is required, in those situations where the catalyst has not been completely damaged beyond recovery.

This is accompanied by an increase in production costs as a result of a higher catalyst requirement, a reduced availability of the plant and a reduced specific performance of the synthesis reactor.

Applicant's experiments with a 200 kW Fischer-Tropsch facility have shown that the operating times of Fischer-Tropsch catalysts may amount to only a few hours to a few days if the above problems have occurred.

The Fischer-Tropsch synthesis is a highly sensitive process. For this reason, changes to parameters, in particular rapid changes thereto, should be avoided as far as possible.

### SUMMARY OF THE INVENTION

It is one object of the invention to avoid operating failures and to thereby increase the availability of a plant for the production of liquid fuels by the Fischer-Tropsch synthesis. It is also an object of the invention to reduce the time for restarting the process and to enhance the specific performance of the synthesis reactor and especially the costs of catalysts for the Fischer-Tropsch synthesis, by means of a more effective operation of the holding operation that has been made necessary by the state of the art.

According to the invention, there is provided a process transitioning a shut down operation into a holding operation in a Fischer-Tropsch synthesis reaction zone employing a catalyst, in response to problems that can lead to a runaway reaction, comprising: discontinuing supply of synthesis gas to the Fischer-Tropsch synthesis reaction zone; and while at least substantially maintaining synthesis reaction pressure and temperature, within a short time after said discontinuing (e.g., preferably not more than about 30 seconds), charging the synthesis reaction zone with inert gas free from catalyst poison, preferably at a volume ratio essentially the same as for the Fischer-Tropsch process, and for a period of time sufficient to flush out reactants from the Fischer-Tropsch reaction zone to at least an extent sufficient to interrupt the synthesis reaction.

Also provided according to the invention is a system for carrying out a Fischer-Tropsch synthesis reaction employing a catalyst, comprising: a Fischer-Tropsch reactor; a control member for discontinuing supply of synthesis gas to the Fischer-Tropsch reactor; a supply of inert gas that is free of catalyst poison, that is at a pressure above the pressure in the Fischer-Tropsch reactor and that is selectively communicable with the reactor; and a control system that is programmed to (1) operate the control member in response to reaction conditions indicative of a runaway reaction, to discontinue the supply of synthesis gas to the reactor, (2) substantially maintain a pressure and temperature in the reactor at levels consistent with the Fischer-Tropsch synthesis reaction, and (3) charge the inert gas into the reactor for a period of time sufficient to flush out reactants from the reactor.

Further objects, features and advantages of the invention will become apparent from the detailed description of preferred embodiments that follows.

### DETAILED DESCRIPTION OF THE PREFERRED EMBODIMENTS

The invention involves carrying out an improved procedure in the case of necessary changes to process parameters, such as those necessary for a shut down operation when problems arise, such that the shut down process becomes a holding operation. In this way, the following effects experienced in the prior art are avoided and/or reduced:
mechanical stress on the catalyst, even up to comminution of the catalyst, as a result of pressure release and the increase in pressure loss accompanying it via the loose catalyst, during subsequent ramping up of the plant in normal operation;
a change in the bulk density of the loose catalyst by rapid temperature decrease;
a reduction of the liquid phase layer required for a controlled Fischer-Tropsch reaction of the catalyst surface;
a high sensitivity of the catalyst after recommissioning of the Fischer-Tropsch synthesis, analogous to initial commissioning;
a relatively long period of time for recommissioning the catalyst, which consequently leads to low production of product;
rapid aging of the catalyst;
a high level of use and high level of performance of flaring that is necessary for pressure reduction of the FT facility; and
relatively high levels of emission at the beginning of every holding operation.

According to the invention, during the necessary change-over from a shut down process to a holding operation of the Fischer-Tropsch synthesis (as a result of problems leading to a runaway reaction of the Fischer-Tropsch reactions) the reactor is not depressurized after cutting off the supply of fresh synthesis gas to the Fischer-Tropsch reactor, and the temperature is not reduced to a temperature below that at which a Fischer-Tropsch reaction no longer takes place. Instead, the reactor is charged, essentially without delay, preferably at least within 30 seconds, with an inert gas, free from catalyst poison, while substantially maintaining the reaction operating conditions. In one preferred embodiment, the inert gas is kept in a vessel at a pressure higher than the operating pressure of the Fischer-Tropsch synthesis, preferably more that two times higher than the operating pressure, wherein the product of pressure and volume is at least about two times the product of pressure and volume of the reaction zone. The inert gas is charged until the reacting components have been flushed out from the FT system via the pressure holding valve. During this process, the reactor is merely inerted for a relatively short time (e.g., a few seconds) sufficient to interrupt the Fischer-Tropsch reaction, while maintaining the pressure and the temperature. After 90 seconds, preferably after 120 seconds, addition of the inert gas is stopped, as well as the gas flow from the Fischer-Tropsch system. In this way, the reactor remains under pressure. Catalyst damage does not occur, and a change to the liquid phase situation on the catalyst is also reduced and/or does not occur, which facilitates the subsequent commissioning phase. It is preferable to limit the decrease in temperature in the Fischer-Tropsch reaction zone to a maximum of 20 K below reaction operating temperature

The invention is illustrated by way of one preferred example.

In the case of a problem which interrupts the gas supply to the reactor, the fresh gas supply system to the Fischer-Tropsch facility is automatically cut off, and a valve is opened simultaneously such that methane or another inert gas from a storage vessel kept at a pressure of up to 200 bar is passed into the feed line to the Fischer-Tropsch reactor, in a volume stream which at least approximately corresponds to the value under normal operation. The inert gas is preferably selected from the group consisting of a noble gas, methane and a mixture of two or more of methane, ethane, ethene and noble gas, which may optionally contain nitrogen, limited to an amount that will not unduly form nitrogen compounds. The inert gas preferably also contains hydrogen in an amount sufficient to ensure maintaining a reducing atmosphere in the FT reactor. The absolute volume of inert gas itself corresponds, in the ideal case, to the volume of the Fischer-Tropsch reactor. The inert gas supply is operated only until the reactants of the Fischer-Tropsch synthesis H₂ and CO are at or below a critical value which, in the ideal case, is zero. In practical operation, the critical value depends on the type of the catalyst and the normal operating temperature of the Fischer-Tropsch reactor.

Following this procedure, a reaction no longer takes place in the Fischer-Tropsch reactor, and no critical situations are able to occur. Depending on the situation which has caused the problem, non-critical parameter changes can subsequently be made, or the pressure and temperature can remain at the prevalent parameters. For example, the temperature in the Fischer-Tropsch reaction zone may be gradually reduced to a value which is still above the temperature needed for starting a Fischer-Tropsch reaction, and/or the pressure in the reaction zone may be gradually reduced to a predetermined level sufficient to avoid damaging the catalyst, e.g., to a level of 80% of the synthesis pressure.

Upon elimination of the problem, the supply of fresh synthesis gas to the reactor can be accomplished after a simplified and more rapid commissioning, since the state of the reaction system (Fischer-Tropsch catalyst/synthesis gas/liquid product on the catalyst) has remained practically unchanged.

Since heat is normally supplied to the reactor during the holding operation, the heat of reaction needs to be discharged during recommissioning. This process is preferably effected automatically by automatic control engineering, such that only the control characteristics need to be taken into account during recommissioning.

The foregoing description of preferred embodiments of the invention has been presented for purposes of illustration and description only. It is not intended to be exhaustive or to limit the invention to the precise form disclosed, and modifications and variations are possible and/or would be apparent in light of the above teachings or may be acquired from practice of the invention. The embodiments were chosen and described in order to explain the principles of the invention and its practical application to enable one skilled in the art to utilize the invention in various embodiments and with various modifications as are suited to the particular use contemplated. It is intended that the scope of the invention be defined by the claims appended hereto and that the claims encompass all embodiments of the invention, including the disclosed embodiments and their equivalents.

What is claimed is:

1. A process for transitioning a shut down operation into a holding operation in a Fischer-Tropsch synthesis reaction zone employing a catalyst, in response to problems that can lead to a runaway reaction, comprising:
discontinuing supply of synthesis gas to the Fischer-Tropsch synthesis reaction zone; and
while at least substantially maintaining synthesis reaction pressure and temperature, within a short time after said discontinuing, charging the synthesis reaction zone with inert gas free from catalyst poison, at a volume ratio essentially the same as for the Fischer-Tropsch process and for a period of time sufficient to flush out reactants from the Fischer-Tropsch reaction zone to an extent sufficient to interrupt the synthesis reaction.

2. A process according to claim 1, wherein said short time is not more than 30 seconds.

3. A process according to claim 1, further comprising, after charging with the inert gas for a period of time of at least 90 seconds, stopping the addition of inert gas and stopping removal of gas from the Fischer-Tropsch reaction zone, to thereby produce a closed system.

4. A process according to claim 3, wherein said period of time is at least 120 seconds.

5. A process according to claim 1, wherein the inert gas is fed in an amount of from 25% to 100% of normal gas stream during operation of the Fischer-Tropsch process.

6. A process according to claim 1, further comprising, after charging with the inert gas, gradually reducing the temperature in the reaction zone to a predetermined level that lies above a level at which the Fischer-Tropsch reaction begins.

7. A process according to claim 6, wherein the temperature is decreased to a maximum of 20 K below reaction operating temperature.

8. A process according to claim 1, further comprising, after charging with the inert gas, gradually reducing the pressure in the reaction zone to a predetermined level.

9. A process according to claim 8, wherein the predetermined pressure level is 80% of normal reaction pressure.

10. A process according to claim 1, wherein the inert gas is selected from the group consisting of a noble gas, methane and a mixture of two or more of methane, ethane, ethene and noble gas, which may optionally contain nitrogen.

11. A process according to claim 10, the inert gas is substantially free of nitrogen, to the extent sufficient to reduce the formation of nitrogen compounds.

12. Process according to claim 1, wherein the inert gas further contains an amount of hydrogen sufficient to ensure a reducing atmosphere in the Fischer-Tropsch reaction zone.

13. A process according to claim 1, wherein the inert gas is supplied from a supply that is at a pressure above the synthesis pressure, wherein the product of pressure and volume is at least about two times the product of pressure and volume of the reaction zone.

14. A process according to claim 1, wherein charging of the inert gas takes place only until H₂ and CO in the Fischer-Tropsch synthesis reaction zone are flushed out from the Fischer-Tropsch reaction zone to a non-critical value for the synthesis reaction.

15. A process according to claim 2, further comprising restarting the Fischer-Tropsch reaction.

16. A system for carrying out a Fischer-Tropsch synthesis reaction employing a catalyst, comprising:
a Fischer-Tropsch reactor;
a control member for discontinuing supply of synthesis gas to the Fischer-Tropsch reactor;
a supply of inert gas that is free of catalyst poison, that is at a pressure above the pressure in the Fischer-Tropsch reactor and that is selectively communicable with the reactor; and
a control system that is programmed to
(1) operate the control member in response to reaction conditions indicative of a runaway reaction, to discontinue the supply of synthesis gas to the reactor,
(2) substantially maintain a pressure and temperature in the reactor at levels consistent with the Fischer-Tropsch synthesis reaction, and
(3) charge the inert gas into the reactor for a period of time sufficient to flush out reactants from the reactor.
